(19) United States
(12) Patent Application Publication
(10) Pub. No.: US 2012/0016263 A1
(43) Pub. Date: Jan. 19, 2012

(54) DEVICE FOR SAMPLING TISSUE FROM AN ANIMAL

(76) Inventors: Jean-Jacques Hilpert, Vitre (FR); Johan Decaluwe, Laval (FR)

(21) Appl. No.: 13/140,877

(22) PCT Filed: Dec. 18, 2009
(86) PCT No.: PCT/EP2009/067590

§ 371 Date: Sep. 29, 2011

(30) Foreign Application Priority Data
Dec. 19, 2008 (FR) 0807262

(51) Int. Cl. A61B 10/00

(52) U.S. Cl. 600/567

(57) ABSTRACT

The invention relates to a device for sampling tissue from an animal, said device cooperating with a sampling and/or marking tool and comprising sampling means provided with at least one cutting element for placing next to an end of a needle-forming element of the tool, and enabling a sample to be taken from the tissue. According to the invention, the sampling means comprise an assembly and protection element arranged in the extension of the cutting element and extending over at least a substantial part of the needle when the sampling means are fixed to the tool.

### FIELD OF THE INVENTION

The field of the invention is that of the control and/or identification of animals.

More specifically, the invention relates to the sampling of animal tissue, enabling in particular the cells with biological or biochemical characteristics of the animal to be preserved, for example in order to subsequently identify the animal or detect diseases in the animal. Such samplings are, for example, performed when placing a marking device, also called a mark.

The invention enables in particular the sample of tissue on bovines, ovines, porcines, caprines, birds, fish or more generally on any animal species.

### PRIOR ART

To improve the monitoring of livestock and guarantee the origin and traceability of animals intended in particular for consumption, one or more tissue samples from the animals concerned are being collected during their life.

A first sample can be taken from the animal at birth, for example when placing an identification mark on the animal. Other samples can be taken subsequently, for example in order to detect diseases or to certify the identity of the animal, by comparison of DNA sequences, and so on.

Once collected, the tissue sample of the animal can be sent directly to a laboratory for analysis. The sample can also be stored for preservation over a number of months to a number of years, by being submerged in a preservative agent, for example.

To facilitate the collection of an animal tissue sample, it is known to use a metal punch capable of being assembled on one of the jaws of a sampling clamp, for example in a removable manner. This punch is classically placed at the end of a rod, rod or plunger secured to a jaw.

Various cutting edge shapes for this punch have been proposed in order to perforate the animal's skin and detach the sample: a substantially circular shape or a more complex shape, in continuous contact or with saw teeth.

It has also been considered to collect the sample when placing an identification mark on the animal (for example on an ear) performed by fitting or snapping a male portion into a female portion.

A technique is known in which the projecting rod of the male portion has a hollow cylindrical passage enabling a cutter, also called the punch, to pass.

Thus, when the male and female portions are fitted together, the rod, rod or plunger of the sampling clamp pushes the punch, which perforates the animal's skin with its cutting edge along a circumferential contact line, while guiding the projecting rod of the male portion into the female portion. The punch is removed after having put the mark in place, and the tissue sample collected remaining inside the punch can be recovered.

A disadvantage of these sampling techniques that may or may not be associated with an animal marking operation is that the sample can become accidentally detached from the punch and be contaminated.

To overcome this problem, a removable punch attached to a first jaw of the sampling tool has been proposed. This punch is fitted at least partially in a tube-shaped container attached to a second jaw of the tool during actuation of the tool, and is detached from the first jaw, thereby enabling it to be removed with the container, for which it can act as a hermetic sealing lid. Owing to this sampling technique, described in document EP 1 014 861, the sample is inserted directly into the container, thus limiting the risk of contamination of the sample.

It is clear, however, that a disadvantage common to the techniques described above lies in the fact that the user must disinfect the punch and the tool after each sampling in order to prevent any contamination of the next animal, which is time-consuming.

Another disadvantage of these known techniques is often the difficulty for the user of assembling the punch on the tool, due to the size of the latter, or, when the weather conditions require it, the wearing of gloves, which makes the user's job difficult.

Yet another disadvantage of these known techniques is that the punch must be handled in order to remove the tissue sample remaining in the central tubular portion of the punch after cutting, in which this latter operation can be difficult and/or complex, and/or present a cutting risk for the user.

There is therefore a need for a new animal tissue sampling technique that does not have all of these disadvantages of the prior art.

### OBJECTIVES OF THE INVENTION

The invention is in particular intended to overcome these various disadvantages of the prior art.

More specifically, the invention is intended to provide a device for collecting an animal tissue sample, which is reliable and risk-free for the animal and the user.

In at least one specific embodiment of the invention, an objective of the invention is to provide a sampling device that does not require decontamination of the tool on which it is assembled, between two samplings.

Another objective of the invention is, in particular, in at least one specific embodiment, to enable a user to perform one or more sequential samplings without having to wear gloves for protection from possible contamination.

Another objective of the invention is to provide a sampling device capable of being used with an existing sampling and/or marking tool.

More specifically, in a particular embodiment, an objective of the invention is to be capable of using such a sampling device with a marking tool already on the market, such as the Total Tagger (registered trademark).

Yet another objective of the invention is to provide a sampling device with simple assembly and disassembly.

### DESCRIPTION OF THE INVENTION

These objectives, as well as others that will appear below, are achieved by means of an animal tissue sampling device that is capable of cooperating with a sampling and/or marking tool, including sampling means intended to be placed on an element forming a rod of said tool, and enabling a sample of said tissue to be detached.

According to the invention, said sampling means include:
an assembly and protection element to be assembled on said rod and for protection of said rod, configured to cover at least a substantial portion of said rod when said sampling means are secured to said tool;
a cutting element, enabling the animal's skin to be perforated when said tool is actuated, arranged in the extension of said assembly and protection element so as to extend beyond the end of said rod.

The invention thus proposes a new approach to the collection of an animal tissue sample, which is particularly simple and fast for the user. In particular, it simplifies the operation of assembly and disassembly on the rod of the tool and makes it possible to no longer have to systematically decontaminate the tool between two sequential samplings, which is doubly advantageous.

According to a particular aspect of the invention, said sampling means include securing means between said cutting element and said assembly and protection element, configured so that said cutting element can be detached from said protection element after said sampling.

Thus, the cutting element can easily be detached from the assembly and protection element after sampling.

Preferably, said assembly and protection element is configured to cooperate with temporary assembly means provided on said tool including at least one element belonging to the group including: a clipping element, a screwing element, or a force-fitted assembly element, and said assembly and protection element remains assembled on said tool after sampling.

Thus, the assembly and protection element is removed with the tool after sampling, without it being necessary to perform an additional operation on the animal, and prevents the risks of contamination of the animal by the user or vice-versa.

According to a particular aspect of the invention, said removable assembly and protection element is configured to be disassembled from said tool by acting on said temporary assembly means and/or on said tool.

This disassembly can in particular be obtained by ejecting the assembly and protection element.

In at least one advantageous embodiment of the invention, at least two distinct portions of said assembly and protection element are secured to said rod, so as to increase the rigidity of said assembly and protection element.

Thus, the assembly and protection element can have rigid portions and more flexible portions, for example in order to facilitate the assembly and/or reduce its weight and therefore its cost, while supporting the cutting element in a suitable manner, and maintaining the satisfactory perforation qualities of the device.

Advantageously, a first of said portions is arranged substantially in the vicinity of the base of said rod-forming element and a second of said portions is arranged at the finer end of said rod-forming element.

Thus, the assembly and protection element is actually assembled on the rod.

Advantageously, said assembly and protection element is made of a biodegradable material.

Thus, once the sampling has been performed, the user can simply dispose of the assembly and protection element, for example by throwing it on the ground, with no harm to the environment.

In at least one embodiment of the invention, said assembly and protection element is made of an at least partially deformable material.

In this way, the assembly on the tool of the assembly and protection element, and the removal thereof, are facilitated.

Advantageously, said assembly and protection element is coated with an antiseptic substance.

Thus, the cleaning of the wound caused by the sampling on the animal is simplified and/or the risks of contamination of the user, animal, or tool by the assembly and protection element are reduced.

In at least one particular embodiment of the invention, said cutting element and said assembly and protection element have a revolution shape with coincident axes.

In particular, in at least one particular embodiment of the invention, the cutting element and/or the assembly and protection element can have a substantially conical shape.

According to a particular aspect of the invention, one of the ends of said assembly and protection element has centring means on said sampling and/or marking device.

Advantageously, said end of said assembly and protection element has an essentially frusto-conical opening capable of cooperating with the base of said element forming a rod.

Advantageously, a sampling device as described above includes storage means designed to receive said cutting element and to detach it from said assembly and protection element.

Thus, the handling of the cutting element is easy and the sample is stored with the cutting element, thereby reducing the risk of contaminating the sample.

In an inventive manner, said cutting element forms means for closing an opening formed in said storage means.

The cutting element can thus act as a hermetic seal for the storage means on which it can be assembled, for example, by fitting, clipping or screwing.

The invention also relates to an assembly and protection element forming an animal tissue sampling device as presented above.

### LIST OF FIGURES

Other features and advantages of the invention will become clear from the following description of a specific embodiment, provided as a simple illustrative and non-limiting example, and the appended drawings, in which:

FIG. 1 shows the sampling means of a sampling device according to an embodiment of the invention, assembled on the rod of a sampling tool in order to collect a sample;

FIG. 2 is a cross-section view of the assembly and protection element of the sampling device shown in FIG. 1;

FIG. 3 shows a storage tube receiving the cutting element of the device shown in FIG. 1, after having assisted in the detachment thereof from the assembly and protection element;

FIGS. 4A and 4B show an example of assembly, before sampling, and disassembly, after sampling, of the assembly and protection element of the device shown in FIG. 1, on the tool, by advantageous temporary assembly means.

### DETAILED DESCRIPTION OF AN EMBODIMENT OF THE INVENTION

### Summary of the Invention

The general principle of the invention is based on the new implementation of sampling means equipped with an active protection of the sampling and/or marking tool on which said sampling means are intended to be mounted. In an indirect manner, the invention also makes it possible to prevent the contamination of the cutting element of the sampling means, as well as of the animal and the user.

The invention thus proposes integrating, with the sampling means, a removable assembly and protection element, in the extension of the cutting element, which advantageously extends over at least a substantial portion of the rod of the sampling and/or marking tool, in order to prevent contact with the animal and/or the sample collected, and therefore to prevent the user from having to decontaminate the tool after each sampling.

At least one embodiment of the invention proposes an assembly and protection element fitting on the rod of a known sampling and/or marking tool. In at least one embodiment of the invention, the assembly and protection element is made of a biodegradable material, for disposal after use.

### Example of an Embodiment of the Invention

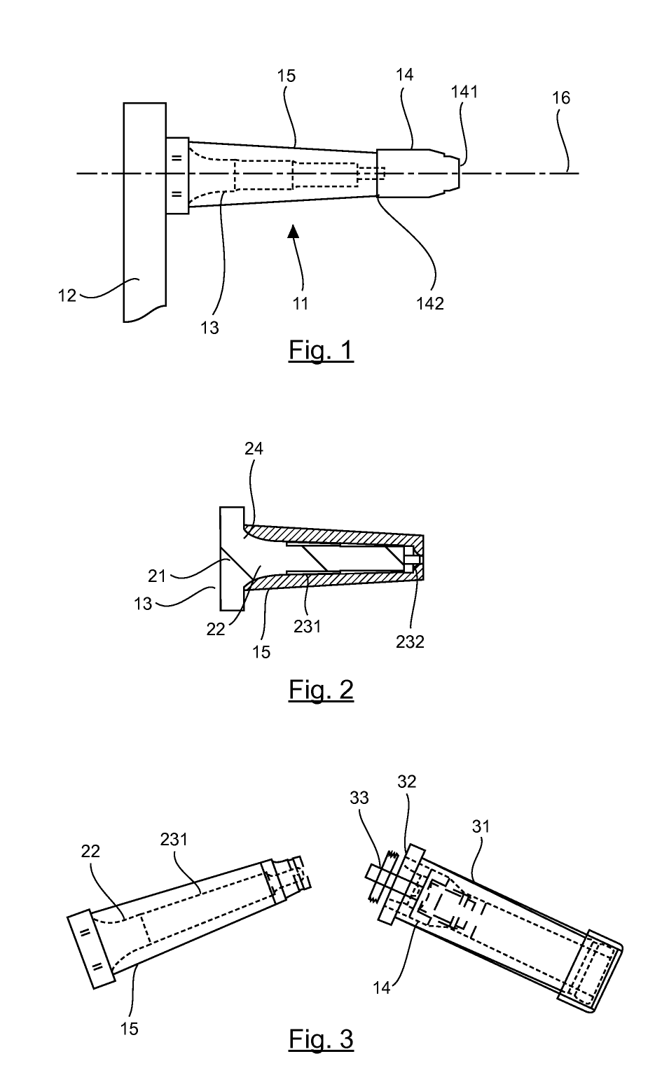

In reference to FIG. 1, an example of an embodiment of the invention in which the animal tissue sampling device according to the invention is used with a known sampling clamp is disclosed.

As shown in FIG. 1, the tissue sampling means 11 according to the invention have been assembled by the user on the clamp 12 in the vicinity of a rod-forming element 13, in order to perform a single sampling.

As already mentioned, the assembly of this device is particularly simple and, as the use of this device makes it possible to eliminate the step of decontamination of the tool between two samplings, a significant acceleration in the sampling speed on a heard is advantageously observed.

The tissue sampling means 11 include a cutting element (“punch”), having a revolution shape, and an assembly and protection element 15 (“sleeve”) arranged in the extension of the cutting element 14, with which it is aligned according to the same axis 16. The respective lengths of the cutting element 14 and the assembly and protection element represent around 70 percent and 30 percent of the total length of the sampling device in this embodiment of the invention.

The cutting element 14 has a cutting edge 141 with a regular circular shape, making it possible to perforate the animal's skin 13 when the clamp 12 is actuated, and means 142 for integration with the assembly and protection device 15 at the base thereof, shown in FIG. 1 in a first state in which the assembly and protection element 15 is secured to the cutting element 14.

In this embodiment, the assembly and protection element 15 or the assembly and protection sleeve, envelops the rod-forming element over substantially the entire length thereof. It has in particular a substantially frusto-conical external shape, to match the general shape of the rod-forming element 13. In alternatives of this embodiment of the invention, other forms of the assembly and protection element can also be envisaged, and can advantageously be chosen from the revolution forms.

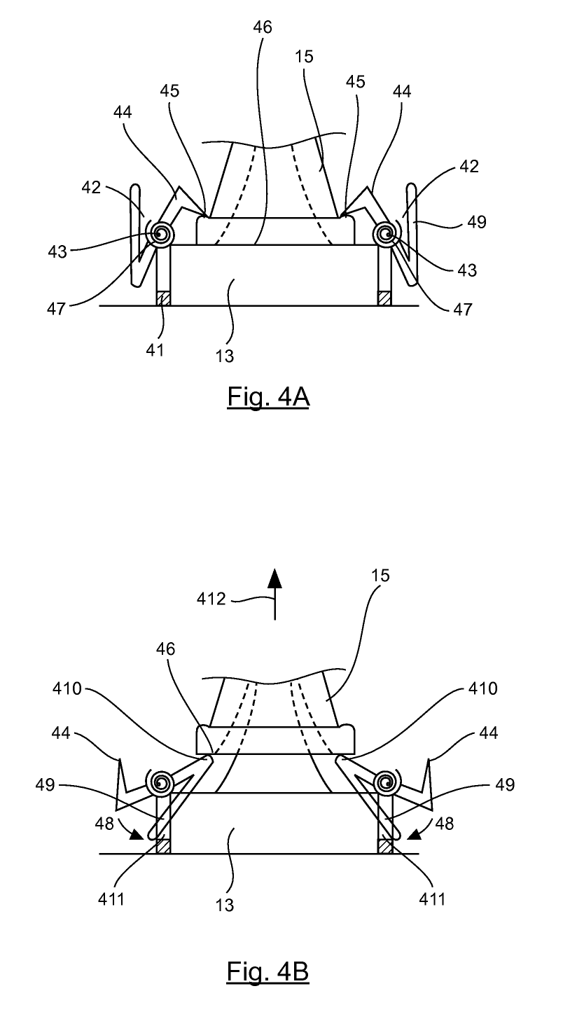

To facilitate the assembly and holding of the assembly and protection element on the sampling tool (for example a clamp), in this embodiment of the invention, in the vicinity of the rod of the later, additional equipment shown in FIG. 4A has been assembled. This equipment has a substantially ring-shaped base 41 on which two levers 42 pivotably connected around their respective axes 43, of which a first arm 44 forms a claw intended to move away from the rod 13 in a first step, then, in a second step, to grip a bulging portion 45 at the base 46 of the assembly and protection element 15 under the action of a return spring 47. These two steps are implemented when the assembly and protection element 15 is progressively pushed in the direction of the base of the rod-forming element 13 to be placed on the sampling tool.

In alternatives of this embodiment of the invention, it is also possible to envisage implementing temporary assembly means on the tool other than the equipment shown in FIG. 4A, such as, for example, one or more clipping and/or screwing and/or force-fitted assembly elements.

It should be noted that the assembly and protection element 15 is made of an at least partially deformable material in order to facilitate its positioning at the base of the rod-forming element 13 and/or the assembly of the assembly and protection element 15 with the base of the cutting element 14.

As can be seen in FIG. 2, in a cross-section drawing, the assembly and protection element 15 includes an internal recess 21 passing through it from one side to the other. This recess 21 has, in the vicinity of the base 46 of the assembly and protection element 15, a substantially bell-shaped flared opening 22 enabling it to be assembled and centred on the base 24 of the rod-forming element 15, followed by an intermediate frusto-conical segment 231 and a tubular end segment 232, with decreasing diameters when moving away from the opening 22, in order to match the shape of the rod. The segment 232 is secured to the finest end of the rod-forming element 13, so as to reinforce and/or increase the rigidity of the assembly and protection element 15.

In alternatives of this embodiment, it is also possible to reduce the thickness of the wall of the assembly and protection element at the level of the intermediate segments, and/or to overlap only a portion of the periphery of the rod with the assembly and protection element.

To be disposed on site by the user after use, the assembly and protection element 15 is made in this embodiment of a biodegradable material, for example from corn or rice. An antiseptic substance is coated on the externals surface of the assembly and protection element 15, in order to clean the animal's wound when the assembly and protection element 15 penetrates with the rod-forming element 13 that it protects, through the circular orifice left by the cutting element 14 when collecting an animal tissue sample.

In this embodiment of the invention, the sampling device includes a storage tool for receiving and helping to detach the cutting element 14. This storage tube is attached on the jaw of the clamp not having the rod-forming element.

When the clamp is actuated, the cutting element 14 is pushed by the assembly and protection element 15 and the rod-forming element 13 into the storage tube, after having cut the tissue sample.

When the clamp is released, the storage tube 31 holds the cutting element 14, of which the base is detached from the assembly and protection element 15, with the latter remaining assembled to the rod-forming element 13, as can be seen in FIG. 3, which shows a second state of the sampling device, after sampling.

The storage tube 31 containing the sample can then be removed from the clamp, then closed again by means, for example, of a hermetic seal 33, fitted or screwed in the opening 32 thereof. The cutting element 14 can also be used as a hermetic seal.

The sample thus stored can be sent to a laboratory for analysis, or preserved for a more or less long time, on the order of several days to several years owing to the effect of a preservative agent present or injected afterward into the storage tube.

It is noted that the storage of tissue samples of an animal makes it possible in particular to subsequently identify the animal or detect diseases in said animal, in view of a microscopic examination or an extraction of a DNA sequence of the sample cells, for example.

FIG. 4B finally shows an example of the disassembly, after sampling, of the assembly and protection element of the device shown in FIG. 1.

As can be seen in FIG. 4B, the assembly and protection element 15 can be removed from the tool after use, by pressing the second arm 49 of the levers 42 in the direction of the arrows 48, causing a rotation movement of the first 44 and second 49 arms of each lever about the respective axis 43 thereof. The bent portion 410 of the second arm 49 of each of the levers 42, after having passed through a passage groove 411, can push on the base 46 of the assembly and protection element and thus eject the assembly and protection element 15 from the tool in the direction of the arrow 412.

### Additional Features and Advantages of the Invention

In at least one other embodiment of the invention, the following can be envisaged:
collecting a tissue sample when placing an auricular mark on an animal, with a sampling device according to the invention cooperating with a marking tool;
a cutting edge of the cutting element with saw teeth;
etc.

1. Animal tissue sampling device configured to cooperate with a sampling and/or marking tool, including sampling means intended to be placed on a rod-forming element of said tool, and enabling a sample of said tissue to be removed,
characterized in that said sampling means include:
an assembly and protection element for assembly on said rod and for protection of said rod, designed to cover at least a substantial portion of said rod when said sampling means are secured to said tool;
a cutting element, enabling the animal's skin to be perforated when said tool is actuated, arranged in the extension of said protection element so as to extend beyond the end of said rod.

2. Sampling device according to claim 1, characterized in that said sampling means include securing means between said cutting element and said assembly and protection element, formed so that said cutting element can be detached from said protection element after said sampling.

3. Sampling device according to claim 2, characterized in that said assembly and protection element is capable of cooperating with temporary assembly means provided on said tool, including at least one element belonging to the group including: a clipping element, a screwing element, or a force-fitted assembly element,
and in that said assembly and protection element remains assembled on said tool after said sampling.

4. Sampling device according to claim 3, characterized in that said removable assembly and protection element is configured so as to be capable of being disassembled from said tool by acting on said temporary assembly means and/or on said tool.

5. Sampling device according to any one of claims 1 to 4, characterized in that at least two distinct portions of said assembly and protection element are secured to said rod, so as to increase the rigidity of said assembly and protection element.

6. Sampling device according to claim 5, characterized in that a first of said portions is arranged substantially in the vicinity of said base of said rod-forming element and a second of said portions is arranged at the finest end of said rod-forming element.

7. Sampling device according to any one of claims 1 to 6, characterized in that said assembly and protection element is made of a biodegradable material.

8. Sampling device according to any one of claims 1 to 7, characterized in that said assembly and protection element is coated with an antiseptic substance.

9. Sampling device according to any one of claims 1 to 8, characterized in that said assembly and protection element is made of an at least partially deformable material.

10. Sampling device according to any one of claims 1 to 9, characterized in that said cutting element and said assembly and protection element have a revolution shape and coincident axes.

11. Sampling device according to any one of claims 1 to 10, characterized in that one of the ends of said assembly and protection element has centring means on said sampling and/or marking device.

12. Sampling device according to claim 11, characterized in that said end of said assembly and protection element has an essentially frusto-conical opening configured to cooperate with the base of said rod-forming element.

13. Sampling device according to any one of claims 1 to 12, characterized in that it includes storage means designed to receive said cutting element and to detach it from said assembly and protection element.

14. Sampling device according to any one of claims 1 to 13, characterized in that said cutting element forms means for closing an opening formed in said storage means.

15. Assembly and protection element of sampling means of an animal tissue sampling device configured to cooperate with a device for sampling and/or marking said animal, in which said sampling means also have at least one cutting element enabling a sample of said tissue to be detached,
characterized in that it is arranged between said cutting element and said sampling and/or marking device when said sampling means are secured to said sampling and/or marking device, so as to prevent bacterial contamination of said animal.
